SEQUENCE LISTING (5 sequences)

SEQ ID NO: 1 - protein, 17 aa, Artificial Sequence
  Synthetic sequence
QLAATKAARAAKTAALQ

SEQ ID NO: 2 - protein, 6 aa, Artificial Sequence
  SYNTHETIC SEQUENCE
QLAATK

SEQ ID NO: 3 - protein, 6 aa, Artificial Sequence
  synthetic sequence
AARAAK

SEQ ID NO: 4 - protein, 5 aa, Artificial Sequence
  synthetic sequence
TAALQ

SEQ ID NO: 5 - protein, 5 aa, Artificial Sequence
  synthetic sequence
LAATK

(19) United States
(12) Patent Application Publication
(10) Pub. No.: US 2015/0355190 A1
(43) Pub. Date: Dec. 10, 2015

(54) Compositions and Methods of Analysis

(71) Applicant: Evol Science LLC, Philadelphia, PA (US)

(72) Inventor: Andrew J. Andrews, Philadelphia, PA (US)

(21) Appl. No.: 14/734,536

(22) Filed: Jun. 9, 2015

Related U.S. Application Data
(60) Provisional application No. 62/009,893, filed on Jun. 9, 2014.
(60) Provisional application No. 62/053,407, filed on Sep. 22, 2014.

(51) Int. Cl. G01N 33/68; C07K 7/08; H01J 49/00

(52) U.S. Cl. CPC G01N 33/6848; H01J 49/0027; C07K 7/08; G01N 2560/00

(57) ABSTRACT

The present disclosure provides compositions and methods for performing analysis on a sample.

### CROSS REFERENCE TO RELATED APPLICATIONS

This application claims priority to U.S. Provisional Application No. 62/009,893, filed Jun. 9, 2014, and U.S. Provisional Application No. 62/053,407, filed Sep. 22, 2014, each of which is hereby incorporated by reference in its entirety.

### BACKGROUND

Analysis of molecular reactions can be complicated due to experimental conditions and the failure to reproduce results due to user error as well as conditions not being optimal for the reaction being investigated. In order to be confident in a result obtained from an experiment it is necessary for an investigator to know that a reaction has been performed properly, under the right conditions, and can be reproduced. In other words, the experiment must be able to be validated. Validation can occur through many methods, but existing methods are time consuming, expensive, and require multiple reagents. Accordingly, there is a need to develop reagents and methods that can be used to validate an experiment. The presently described subject matter fulfills these needs as well as others.

### SUMMARY OF THE INVENTION

In some embodiments, methods of validating a reaction of a test sample are provided. In some embodiments, the method comprises reacting a test sample comprising a pH-indicating agent, a molecule of interest and a Multiplexed Control and Relative Quantitation (“MCRQ”) standard, which can also be referred to as a standard or “Quantitative Multiplexed Control” (“QMC”), with a propionylating agent and a digesting agent; and introducing the reacted sample into a mass spectrometer, wherein if one or more peaks produced in the mass spectrometer attributed to the QMC are above a selected threshold the reaction is validated for the reacting step. Thus, in some embodiments, the QMC can be used to validate and/or track enzymatic or chemical modifications to substrate molecules using mass spectrometry as described herein.

In some embodiments, methods of cross-validating a plurality of reactions, the method comprising performing a first reaction, the first reaction comprising reacting a first test sample comprising a pH-indicating agent, a molecule of interest and a quantitative multiplexed control (QMC) with a propionylating agent and/or a digesting agent; performing a second reaction, the second reaction comprising reacting a second test sample comprising a pH-indicating agent, a molecule of interest and a quantitative multiplexed control (QMC) with a propionylating agent and/or a digesting agent; performing a first mass spectrometry run with the first reaction and a second mass spectrometry run with the second reaction; calculating a Q-ratio of the QMC of the first reaction and a Q-ratio of the QMC of the second reaction; wherein if the Q-ratio of the first reaction and the Q-ratio of the second reaction are substantially the same the first and second reactions are cross-validated; or wherein if the Q-ratio of the first reaction and the Q-ratio of the second reaction are not substantially the same the first and second reactions are not cross-validated.

In some embodiments, a kit for performing a method is provided herein. In some embodiments, the kit includes a QMC, a pH-indicating agent, a propionylating agent and/or a digesting agent, a base, and optionally an extraction buffer, a quenching reagent, ammonium bicarbonate, or any combination thereof. In some embodiments, the kit does not comprise a pH-indicating agent.

In some embodiments, a QMC is provided. In some embodiments, the QMC is a peptide.

### BRIEF DESCRIPTION FIGURES

FIG. 1 illustrates the various fragments generated from a non-limiting example of an embodiment disclosed herein.

FIG. 2 illustrates an example of quantitative mass spectrometry data from histone sample treatments normalized by the QMC.

### DETAILED DESCRIPTION

This description is not limited to the particular processes, compositions, or methodologies described, as these may vary. The terminology used in the description is for the purpose of describing the particular versions or embodiments only, and it is not intended to limit the scope of the embodiments described herein. Unless defined otherwise, all technical and scientific terms used herein have the same meanings as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. However, in case of conflict, the patent specification, including definitions, will prevail.

It must also be noted that as used herein and in the appended claims, the singular forms “a”, “an”, and “the” include plural reference unless the context clearly dictates otherwise.

As used in this document, terms “comprise,” “have,” and “include” and their conjugates, as used herein, mean “including but not limited to.” While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used herein, the term “about” refers to a value that is ±10% of the value that the term “about” is modifying. When the term “about” is used to modify a list or range, each member of that is list is modified by the term “about” even if the term “about” is not recited before each member of the list individually unless context explicitly dictates otherwise. For example, the phrase “about 10, 20, or 30” should be understood to mean “about 10, about 20, or about 30,” unless context explicitly dictates otherwise. For example, the phrase “about 10 to 20” should be understood to mean “about 10 to about 20,” unless context explicitly dictates otherwise.

Embodiments disclosed herein provide methods of validating chemical and/or biological reactions. The validation can be used to confirm that a reaction has occurred and to quantify the reaction's efficiency and completeness status. These results can be used to provide confidence to an individual or user of a method that the results of the reaction are correct, i.e., valid. Reactions are performed with an internal reference/standard that is also acted upon during the reaction in concert with the experimental substrate. The standard/reference can be used to evaluate whether the reaction has taken place, to what state of completeness, and how efficiently. The standard can be referred to as a Quantitative Multiplexed Control (QMC) because a single molecule can provide details (e.g. validity, efficiency, and completion) about steps of an experiment. This information increases the reproducibility of experiments and allows for quantitation, and standardization of experiments with a single molecule, such as the QMC. Prior to the presently disclosed subject matter there would not have been an expectation of success or prediction that a single molecule would be able to perform multiple functions. The single molecule can also be used to determine whether multiple steps of a reaction are being performed correctly. For example, if sample is being treated with a propionylating agent and a digesting agent (e.g. protease) the QMC will provide data regarding the two different reactions (e.g., propionylation and digestion) because of, for example, the structure of the standard. In this non-limiting example, the QMC will have a structure such that it will be propionylated and digested. These fragments and modifications can be analyzed by, for example, mass spectrometry. Since the standard is present in known amounts, mass spectra produced can be analyzed to determine the efficiency and completion of the reactions. The standard can also be used to quantify molecule(s) of interest in the sample. This can be done by comparing peak intensities of the standard to peaks attributed to the molecule(s) of interest measured with the mass spectrometer. In some embodiments, in each sample, peak intensities of the molecule(s) of interest can be divided by the peak intensities of the standard to obtain Q-ratios of individual peaks of interest.

Additionally, in some embodiments, the sum of the intensity (or signal) from all forms of the QMC (initial, modified only (e.g. post-translational modification), digested only (cleavage event), and/or modified and digested) can be used as an internal standard which can be used to quantitate targets of interest between samples and experiments. This can be referred to as a Q-ratio or the ion (peptide or modification divided by the sum of the QMC states). In some embodiments, the peptide is not modified and/or digested. The Q-ratio can be calculated based upon the different states that exist in the reaction and the states do not have to include all of the options listed above. That is, in some embodiments, it can be one or more of those listed herein and above. This can be used for quantitation and to compare sample handling between samples or experiments as well as to compare different machine parameters or machines. A non-limiting example of this is described in Example 2. Accordingly, in some embodiments, the QMCs described herein can be used to compare experimental conditions to one another as well as to compare experiments that are done at different times, by different individuals, and the like. For example, if the Q-ratio is the same Because of the characteristics of the QMC this is possible because it is based upon a single molecule (e.g. peptide) that can be monitored by mass spectrometry that shows the amount of the unmodified (initial), modified (e.g. propionylated), and digested (e.g. cleaved at a arginine residue). Accordingly, the QMC represents a significant and unexpected improvement over prior standards and controls that have previously been used. Prior to the presently described embodiments, there was not a single molecule that could perform all of the functions described herein.

Accordingly, in some embodiments, a multiplexed control and relative quantitation standard is provided. The multiplexed control and relative quantitation standard is a single molecule. The structure of the molecule can vary, but should have groups that react in the manner that a molecule of interest will react in a reaction. For example, if a molecule of interest will be propionylated then the QMC should be able to be propionylated. Another non-limiting example is if the molecule of interest is to be phosphorylated or dephosphorylated the QMC should also be able to be phosphorylated or dephosphorylated. In this manner, the standard can be used to evaluate the efficiency of the reaction. If the reaction on the standard is not complete or mostly complete then the user knows that the reaction is not valid and the conditions should be altered. The validity of the reaction can be analyzed through the use of mass spectrometry since the reactions can be monitored and measured accurately through the use of mass spectrometry (e.g. LC-MS). In some embodiments, the QMC is a peptide. In some embodiments, the peptide is, or comprises a sequence of, QLAATKAARAAKTAALQ (SEQ ID NO: 1). The QMC can also provide controls for sample loading differences and quantities, chemical derivatization, digestion, and C-18 elution time, and provide a reference point for normalization and relative quantitation. All of these controls and references are performed by one molecule. In some embodiments, the QMC is a polymer (e.g. an amino acid sequence; initial or product ion). In some embodiments, the QMC is not found in the target sample. The QMC will, or should, contain reactive groups specific for the chemical modification and these sites must, or should, also be digested by a digesting agent or disassociated in the mass spectrometer either before or after the reactive groups. The simplest of these groups would be a lysine, which can be reacted with propionic anhydride that prevents the digestion of the peptide by trypsin. This allows for different read outs, for example, 1) derivatization worked but digestion failed and 2) digestion worked but derivatization failed. In some embodiments, the standard can have a retention time on C-18 that is appropriate for the experiment. The standard should be large enough for good detection by the instrument in use. While the size is determined by the mass spectrometer in use it should be small enough that the pre-digested fragment can be measured and the resulting digested fragments are not too small to be measured accurately. The length of the polymer can be designed based upon the needs of the individual performing the method.

The QMC can also comprise a plurality of domains, wherein the domains are separated by one or more digestion sites. The plurality of domains can be used to quantify the absolute amount of a molecule of interest. For example, a QMC with domains A and B can have twice as many copies of domain B as compared to domain A. When the QMC is digested with the digesting agent it will have a total peak intensity for domain B that is twice as much as domain A. Because the exact amount of the QMC included in the reaction is known, the ratio of the peak intensity of domain B to domain A can be used to determine the absolute concentration of the molecule of interest in the test sample. The QMC, which can be a peptide, can have one or more domains. In some embodiments, the domains are not in a 1:1 ratio. In some embodiments, the domains are separated by a lysine, arginine, or other modifiable residue, such as threonine, serine, and the like. The lysine, can for example, be propionylated. The arginine can be used as a cleavage site. The domains can also be separated by other residues that are capable of being post-translationally modified. For example, serine and threonine can be phosphorylated or dephosphorylated. If the reaction involves dephosphorylation, the QMC can be phosphorylated before being reacted in a test sample. In some embodiments, the QMC has both a propionylation site and/or a cleavage site. In some embodiments, the QMC has only a propionylation site or only a cleavage site. In some embodiments, the QMC comprises a first domain, a second domain, and a third domain. In some embodiments, the first, second, and third domains, are separated by a post-translational modification site. In some embodiments, it is a propionylation site. In some embodiments, the first, second, and third domains, are separated by a digestion (cleavage) modification site, such as, but not limited to, an arginine. The digestion site can also be a more specific recognition sequence that is specific for a specific protease.

In some embodiments, the QMC is about 8 to about 20, about 10 to about 20, about 12 to about 20, about 14 to about 20, about 16 to about 20, about 18 to about 20, about 8 to about 18, about 10 to about 16, about 8 to about 14, about 8 to about 12, about 8 to about 10, about 9 to about 20, about 9 to about 18, about 9 to about 16, about 9 to about 14, about 9 to about 12, about 9 to about 11, about 9 to about 13, about 10 to about 20, about 10 to about 18, about 10 to about 16, about 10 to about 14, about 10 to about 13, about 10 to about 12, about 11 to about 20, about 11 to about 18, about 11 to about 16, about 11 to about 14, about 11 to about 13, about 12 to about 20, about 12 to about 18, about 12 to about 16, about 12 to about 14, about 13 to about 20, about 13 to about 18, about 13 to about 16, about 13 to about 15, about 14 to about 20, about 14 to about 18, about 14 to about 16, about 20 to about 25, about 16 to about 22, about 18 to about 22 residues.

In some embodiments, the QMC is a peptide sequence, with a length as described herein, or in a range as described herein, that is not found in nature. For example, the QMC sequence described in Example 1 is not known to exist in nature, which assists in the analysis of the reaction because there is less risk that the peaks identified in the mass spectra will overlap with what is in the reaction sample. The QMC can exist in nature, but in some embodiments it does not.

As described herein, the QMC can be made up of domains. In some embodiments, the domains can be a tripeptide.

In some embodiments, the QMC has a formula of: R₁-X₁-R₂-X₂-R₃-X₃-R₄-X₄-R₅, wherein R₁, R₂, R₃, R₄, and R₅ are each independently a tripeptide or null provided that no more than two of R₁, R₂, R₃, R₄, and R₅ are null; and X₁, X₂, X₃, and X₄ are each independently null, lysine, arginine, or another residue that can be post-translationally modified (e.g. serine or threonine). In some embodiments, the tripeptide is a peptide that is not known to be found in nature. In some embodiments one of X₁, X₂, X₃, and X₄ are independently lysine or arginine and the remaining are null. In some embodiments two of X₁, X₂, X₃, and X₄ are independently lysine or arginine and the remaining are null. In some embodiments three of X₁, X₂, X₃, and X₄ are independently lysine or arginine and the remaining is null. In some embodiments, each of X₁, X₂, X₃, and X₄ are independently lysine or arginine. In some embodiments, X₁ is lysine or arginine and X₂, X₃, and X₄ are null. In some embodiments, X₂ is lysine or arginine and X₁, X₃, and X₄ are null. In some embodiments, X₃ is lysine or arginine and X₁, X₂, and X₄ are null. In some embodiments, X₄ is lysine or arginine and X₁, X₂, and X₃ are null. In some embodiments, X₁ and X₂ are each independently lysine or arginine and X₃ and X₄ are null. In some embodiments, X₁ and X₃ are each independently lysine or arginine and X₂ and X₄ are null. In some embodiments, X₂ and X₃ are each independently lysine or arginine and X₁ and X₄ are null. In some embodiments, X₃ and X₄ are each independently lysine or arginine and X₁ and X₂ are null. In some embodiments, X₁ and X₄ are each independently lysine or arginine and X₂ and X₃ are null. In some embodiments, X₁, X₂, and X₃ are each independently lysine or arginine and X₄ is null. In some embodiments, X₁, X₂, and X₄ are each independently lysine or arginine and X₃ is null. In some embodiments, X₂, X₃, and X₄ are each independently lysine or arginine and X₁ is null. In some embodiments, X₁, X₃, and X₄ are each independently lysine or arginine and X₂ is null. In some embodiments, X₁, X₂, X₃, and X₄ are null.

In some embodiments, the QMC has a formula of: R₁-X₁-R₂-X₂-R₃-X₃-R₄, wherein R₁, R₂, R₃, and R₄, are each independently a tripeptide; and X₁, X₂, and X₃ are each independently null, lysine, arginine, or another residue that can be post-translationally modified (e.g. serine or threonine). In some embodiments, the tripeptide is a peptide that is not known to be found in nature. In some embodiments one of X₁, X₂, and X₃ are lysine or arginine and the remaining are null. In some embodiments two of X₁, X₂, and X₃ are independently lysine or arginine and the remaining is null. In some embodiments each of X₁, X₂, and X₃ is independently lysine or arginine. In some embodiments, X₁ is lysine or arginine and X₂ and X₃ are null. In some embodiments, X₂ is lysine or arginine and X₁ and X₃ is null. In some embodiments, X₃ is lysine or arginine and X₁ and X₂ are null. In some embodiments, X₁ and X₂ are each independently lysine or arginine and X₃ is null. In some embodiments, X₁ and X₃ are each independently lysine or arginine and X₂ is null. In some embodiments, X₂ and X₃ are each independently lysine or arginine and X₁ is null. In some embodiments, each of X₁, X₂, and X₃ is null.

In some embodiments, the QMC has a formula of: R₁-X₁-R₂-X₂-R₃, wherein R₁, R₂, and R₃, are each independently a tripeptide; and X₁ and X₂ are each independently null, lysine, arginine, or another residue that can be post-translationally modified (e.g. serine or threonine). In some embodiments, the tripeptide is a peptide that is not known to be found in nature. In some embodiments one of X₁ and X₂ is null. In some embodiments, both are null. In some embodiments one of X₁ and X₂ is lysine and the other is arginine. In some embodiments, both are lysine or both are arginine.

In some embodiments, R₁, R₂, R₃, R₄, and R₅ of the various formula described herein are each independently a tripeptide comprising only L-amino acid residues. In some embodiments, R₁, R₂, R₃, R₄, and R₅ are each independently a tripeptide comprising only D-amino acid residues. In some embodiments, R₁, R₂, R₃, R₄, and R₅ can be the same tripeptide or different tripeptides. In some embodiments, the tripeptide is a mixture of D- and L-amino acid residues. In some embodiments, the tripeptide is not a tripeptide found in nature.

As used herein “found in nature” refers to whether the 3 amino acid sequence exists in a peptide known to be in nature. This analysis can be done, for example, doing a BLASTP search at the NCBI website using default settings and searching the non-redundant database (nr).

In some embodiments, R₁, R₂, R₃, R₄, and R₅ are each independently selected from the group of peptides listed in Table 1 and/or Table 2. In some embodiments, R₁, R₂, R₃, R₄, and R₅ of the various formula described herein are each independently selected from the group of peptides listed in Table 1.

TABLE 1

| | | | | | | |
|---|---|---|---|---|---|---|
| NEI | DIY | QMS | GFW | ITV | SSV | AEF |
| NEM | DIV | QMT | GFY | IWW | STT | AEP |
| NEF | DLL | QMY | GFV | IWY | STW | AES |
| NES | DLM | QMV | GPP | IWV | STY | AET |
| NEW | DLF | QFF | GPY | IYY | STV | AEW |
| NEY | DLP | QFS | GPV | IYV | SWW | AEY |
| NGG | DLS | QFT | GSW | IVV | SWY | AEV |
| NGH | DLT | QFW | GTT | LLL | SWV | AGG |
| NGI | DLW | QFY | GTW | LLM | SYY | AGH |
| NGL | DLY | QFV | GTY | LLF | SYV | AGI |
| NGW | DLV | QPY | GTV | LLP | TTT | AGL |
| NHH | DMM | QSW | GWW | LMM | TTW | AGM |
| NHL | DMF | QSY | GYY | LMF | TTY | AGF |
| NHF | DMP | QSV | GYV | LMP | TTV | AGP |
| NHP | DMS | QTT | GVV | LMS | TWW | AGS |
| NHW | DMT | QTW | HHH | LMT | TWY | AGT |
| NHY | DMW | QTY | HHI | LMW | TWV | AGW |
| NHV | DMY | QTV | HHL | LMY | TYY | AGY |
| NIL | DMV | QWW | HHK | LMV | TVV | AGV |
| NIM | DFF | QWY | HHM | LFY | WWW | AHH |
| NIF | DFP | QWV | HHF | LPP | WWY | AHL |
| NIS | DFS | QYY | HHP | LPY | WWV | AHM |
| NIT | DFT | QYV | HHS | LSS | WYY | AHF |
| NIW | CQT | QVV | HHT | LST | WYV | AHS |
| NIY | CQY | EEE | HHW | LSW | WVV | AHW |
| NIV | CGT | EEG | HHY | LSY | YYY | AII |
| NLL | CIM | EEH | HHV | LSV | YYV | AIL |
| NLM | CIW | EEI | HII | LTT | YVV | AIM |
| NLF | CMM | EEL | HIL | LTW | VVV | AIF |
| NFS | CMF | EEM | HIM | LTV | AAA | AIP |
| NFW | CMP | EEF | HIP | LWW | AAN | AIS |
| NFY | CMS | EEP | HIS | LWY | AAD | AIW |
| NFV | CMT | EES | HIT | LWV | AAC | AIY |
| NPP | CMW | EET | HIW | LYY | AAQ | AMM |
| NPS | CMV | EEW | HIY | LVV | AAE | AMF |
| NTW | CFF | EEY | HIV | MMM | AAG | AMP |
| NTY | CFP | EGL | HLL | MMF | AAH | AMS |
| NWW | CFS | EGF | HLK | MMP | AAI | AMW |
| NWY | CFW | EHF | HLM | MMS | AAL | AMY |
| NYY | CFY | EHP | HLF | MMT | AAM | AMV |
| NYV | CPP | EHW | HLP | MMW | AAF | AFF |
| NVV | CPT | EHV | HLS | MMY | AAP | AFP |
| DDD | CPW | EIM | HLT | MMV | AAS | AFS |
| DDC | CPY | EIF | HLW | MFF | AAT | AFT |
| DDQ | CSS | EIP | HMV | MFP | AAW | AFW |
| DDE | CST | EIS | HFF | MFS | AAY | AFY |
| DDG | CSW | EIW | HFP | MFT | AAV | AFV |
| DDI | CSY | EIY | HFS | MFW | ANN | APP |
| DDL | CSV | ELL | HFW | MFY | AND | APW |
| DDM | CTW | ELM | HFY | MFV | ANC | APY |
| DDF | CTY | ELW | HPP | MPP | ANQ | ASW |
| DDP | CTV | ELY | HPS | MPS | ANE | ASY |
| DDT | CWW | EMM | HPW | MPW | ANG | ASV |
| DDW | CWY | EMF | HPY | MPY | ANH | ATT |
| DDY | CWV | EMT | HSW | MPV | ANI | ATW |
| DDV | CYY | EMW | HSY | MSS | ANL | ATY |
| DCC | CYV | EMY | HTT | MST | ANM | AWW |
| DCQ | CVV | EFF | HTW | MSW | ANF | AWY |
| DCE | QQQ | EFP | HTY | MTW | ANP | AWV |
| DCG | QQE | EFS | HTV | MTY | ANS | AYY |
| DCH | QQG | EFT | HWW | MTV | ANT | AYV |
| DCP | QQH | EFW | HWY | MWW | ANW | AVV |
| DCS | QQI | EFY | HWV | MWY | ANY | NNN |
| DCW | QQL | EFV | HYY | MWV | ANV | NND |
| DCY | QQM | EPP | HYV | MYY | ADD | NNC |
| DCV | QQF | ESW | HVV | MYV | ADC | NNQ |
| DQQ | QQP | ETY | III | MVV | ADQ | NNE |
| DQG | QQT | EWW | IIL | FFF | ADE | NNG |
| DQH | QQW | EWY | IIM | FFP | ADG | NNH |
| DQI | QQY | EWV | IIF | FFW | ADH | NNI |
| DQM | QQV | EYY | IIP | FFY | ADI | NNL |
| DQT | QEE | EYV | IIS | FFV | ADL | NNM |
| DQW | QEG | EVV | IIT | FPP | ADM | NNF |
| DEE | QEH | GGG | IIW | FPW | ADF | NNP |
| DEH | QEM | GGH | IIY | FPY | ADP | NNS |
| DEF | QEF | GGI | IIV | FPV | ADS | NNT |
| DEW | QEW | GGL | ILL | FSS | ADT | NNW |
| DEY | QEY | GGM | ILM | FST | ADW | NNY |
| DEV | QGG | GGF | ILF | FSW | ADY | NNV |
| DGG | QGH | GGP | ILP | FSY | ADV | NDD |
| DGH | QGF | GGS | ILS | FSV | ACC | NDC |
| DGI | QGP | GGT | ILT | FTT | ACQ | NDL |
| DGL | QGS | GGW | ILW | FTW | ACE | NDM |
| DGM | QGW | GGY | ILY | FTY | ACH | NDF |
| DGF | QGY | GGV | ILV | FTV | ACI | NDT |
| DGP | QGV | GHH | IMF | FWW | ACL | NDW |
| DGS | QHH | GHW | IMP | FWY | ACM | NCC |
| DGT | QHM | GHV | IMS | FWV | ACF | NCQ |
| DGW | QHS | GII | IMT | FYY | ACP | NCE |
| DGY | QHT | GIL | IMW | FYV | ACS | NCG |
| DGV | QHW | GIM | IMY | FVV | ACT | NCH |
| DHH | QHY | GIF | IMV | PPP | ACW | NCI |
| DHI | QHV | GIP | IFF | PPS | ACY | NCL |
| DHL | QII | GIT | IFP | PPT | AQQ | NCM |
| DHM | QIL | GIW | IFS | PPW | AQH | NCF |
| DHF | QIM | GIY | IFT | PPY | AQI | NCP |
| DHP | QIF | GLL | IFW | PPV | AQL | NCT |
| DHS | QIP | GLM | IFY | PSW | AQM | NCW |
| DHT | QIT | GLF | IFV | PSY | AQF | NCY |
| DHW | QIW | GLP | IPP | PSV | AQP | NCV |
| DHY | QIY | GLS | IPW | PTW | AQS | NQQ |
| DHV | QIV | GLW | IPY | PTY | AQT | NQE |
| DII | QLM | GMM | ISS | PWW | AQW | NQG |
| DIL | QLT | GMF | IST | PWY | AQY | NQH |
| DIM | QLW | GMS | ISW | PWV | AQV | NQI |
| DIF | QLY | GMT | ISY | PYY | AEG | NQF |
| DIP | QLV | GMW | ISV | PYV | AEH | NQW |
| DIS | QMM | GMY | ITT | SSS | AEI | NQY |
| DIT | QMF | GFF | ITW | SSW | AEL | NEG |
| DIW | QMP | GFP | ITY | SSY | AEM | NSW |
| NSV | NTT | | | | | |

TABLE 2

| | | | | | | |
|---|---|---|---|---|---|---|
| NPW | DST | CCE | CQG | CEK | CGP | CHV |
| NPY | DSW | CCG | CQH | CEM | CGS | CII |
| NPV | DSY | CCH | CQI | CEF | CGW | CIL |
| NSS | DSV | CCI | CQL | CEP | CGY | CIK |
| NST | DTT | CCL | CQK | CES | CGV | CIF |
| NSY | DTW | CCK | CQM | CET | CHH | CIP |
| DFW | DTY | CCM | CQF | CEW | CHI | CIS |
| DFY | DTV | CCF | CQP | CEY | CHL | CIT |
| DFV | DWW | CCP | CQS | CEV | CHK | CIY |
| DPP | DWY | CCS | CQW | CGG | CHM | CIV |
| DPS | DWV | CCT | CQV | CGH | CHF | CLL |
| DPT | DYY | CCW | CEE | CGI | CHP | CLK |
| DPW | DYV | CCY | CEG | CGL | CHS | CLM |
| DPY | DVV | CCV | CEH | CGK | CHT | CLF |
| DPV | CCC | CQQ | CEI | CGM | CHW | CLP |
| DSS | CCQ | CQE | CEL | CGF | CHY | CLS |
| CLT | CLW | CLY | CLV | | | |

In some embodiments, R₁, R₂, R₃, R₄, and R₅ of the various formula described herein are not the tripeptides listed in Table 2. In some embodiments, R₁, R₂, R₃, R₄, and R₅ of the various formula described herein are not the tripeptides listed in Table 3.

TABLE 3

| | | | | | | |
|---|---|---|---|---|---|---|
| DGK | EII | HMY | KYV | DQS | RRT | RGT |
| DHK | EIL | HFT | KVV | DQY | RRW | RGW |
| DIK | EIK | HFV | MPT | DQV | RRY | RGY |
| DLK | EIT | HPT | MSY | DEG | RRV | RGV |
| DKK | EIV | HPV | MSV | DEI | RNN | RHH |
| DKM | ELK | HSS | MTT | DEL | RND | RHI |
| DKF | ELF | HST | FFS | DEK | RNC | RHL |
| DKP | ELP | HSV | FFT | DEM | RNQ | RHK |
| DKS | ELS | IIK | FPS | DEP | RNE | RHM |
| DKT | ELT | ILK | FPT | DES | RNG | RHF |
| DKW | ELV | IKK | PSS | DET | RNH | RHP |
| DKY | EKK | IKM | PST | AAR | RNI | RHS |
| DKV | EKM | IKF | PTT | AAK | RNL | RHT |
| CKK | EKF | IKP | PTV | ARR | RNK | RHW |
| CKM | EKP | IKS | PVV | ARN | RNM | RHY |
| CKF | EKS | IKT | SST | ARD | RNF | RHV |
| CKP | EKT | IKW | SVV | ARC | RNP | RII |
| CKS | EKW | IKY | TYV | ARQ | RNS | RIL |
| CKT | EKY | IKV | NCS | ARE | RNT | RIK |
| CKW | EKV | IMM | NQL | ARG | RNW | RIM |
| CKY | EMP | IPS | NQK | ARH | RNY | RIF |
| CKV | EMS | IPT | NQM | ARI | RNV | RIP |
| CMY | EMV | IPV | NQP | ARL | RDD | RIS |
| CFT | EPS | LLK | NQS | ARK | RDC | RIT |
| CFV | EPT | LLS | NQT | ARM | RDQ | RIW |
| CPS | EPW | LLT | NQV | ARF | RDE | RIY |
| CPV | EPY | LLW | NEE | ARP | RDG | RIV |
| CTT | EPV | LLY | NEH | ARS | RDH | RLL |
| QQK | ESS | LLV | NEL | ART | RDI | RLK |
| QQS | EST | LKK | NEK | ARW | RDL | RLM |
| QEI | ESY | LKM | NEP | ARY | RDK | RLF |
| QEL | ESV | LKF | NET | ARV | RDM | RLP |
| QEK | ETT | LKP | NEV | ANK | RDF | RLS |
| QEP | ETW | LKS | NGK | ADK | RDP | RLT |
| QES | ETV | LKT | NGM | ACG | RDS | RLW |
| QET | GGK | LKW | NGF | ACK | RDT | RLY |
| QEV | GHI | LKY | NGP | ACV | RDW | RLV |
| QGI | GHL | LKV | NGS | AQE | RDY | RKK |
| QGL | GHK | LFF | NGT | AQG | RDV | RKM |
| QGK | GHM | LFP | NGY | AQK | RCC | RKF |
| QGM | GHF | LFS | NGV | AEE | RCQ | RKP |
| QGT | GHP | LFT | NHI | AEK | RCE | RKS |
| QHI | GHS | LFW | NHK | AGK | RCG | RKT |
| QHL | GHT | LFV | NHM | AHI | RCH | RKW |
| QHK | GHY | LPS | NHS | AHK | RCI | RKY |
| QHF | GIK | LPT | NHT | AHP | RCL | RKV |
| QHP | GIS | LPW | NII | AHT | RCK | RMM |
| QIK | GIV | LPV | NIK | AHY | RCM | RMF |
| QIS | GLK | LTY | NIP | AHV | RCF | RMP |
| QLL | GLT | LYV | NLK | AIK | RCP | RMS |
| QLK | GLY | KKK | NLP | AIT | RCS | RMT |
| QLF | GLV | KKM | NLS | AIV | RCT | RMW |
| QLP | GKK | KKF | NLT | ALL | RCW | RMY |
| QLS | GKM | KKP | NLW | ALK | RCY | RMV |
| QKK | GKF | KKS | NLY | ALM | RCV | RFF |
| QKM | GKP | KKT | NLV | ALF | RQQ | RFP |
| QKF | GKS | KKW | NKK | ALP | RQE | RFS |
| QKP | GKT | KKY | NKM | ALS | RQG | RFT |
| QKS | GKW | KKV | NKF | ALT | RQH | RFW |
| QKT | GKY | KMM | NKP | ALW | RQI | RFY |
| QKW | GKV | KMF | NKS | ALY | RQL | RFV |
| QKY | GMP | KMP | NKT | ALV | RQK | RPP |
| QKV | GMV | KMS | NKW | AKK | RQM | RPS |
| QMW | GFS | KMT | NKY | AKM | RQF | RPT |
| QFP | GFT | KMW | NKV | AKF | RQP | RPW |
| QPP | GPS | KMY | NMM | AKP | RQS | RPY |
| QPS | GPT | KMV | NMF | AKS | RQT | RPV |
| QPT | GPW | KFF | NMP | AKT | RQW | RSS |
| QPW | GSS | KFP | NMS | AKW | RQY | RST |
| QPV | GST | KFS | NMT | AKY | RQV | RSW |
| QSS | GSY | KFT | NMW | AKV | REE | RSY |
| QST | GSV | KFW | NMY | AMT | REG | RSV |
| EEK | GWY | KFY | NMV | APS | REH | RTT |
| EEV | GWV | KFV | NFF | APT | REI | RTW |
| EGG | HIK | KPP | NFP | APV | REL | RTY |
| EGH | HIF | KPS | NFT | ASS | REK | RTV |
| EGI | HLY | KPT | NPT | AST | REM | RWW |
| EGK | HLV | KPW | NTV | ATV | REF | RWY |
| EGM | HKK | KPY | NWV | RRR | REP | RWV |
| EGP | HKM | KPV | DDH | RRN | RES | RYY |
| EGS | HKF | KSS | DDK | RRD | RET | RYV |
| EGT | HKP | KST | DDS | RRC | REW | RVV |
| EGW | HKS | KSW | DCI | RRQ | REY | NNK |
| EGY | HKT | KSY | DCL | RRE | REV | NDQ |
| EGV | HKW | KSV | DCK | RRG | RGG | NDE |
| EHH | HKY | KTT | DCM | RRH | RGH | NDG |
| EHI | HKV | KTW | DCF | RRI | RGI | NDH |
| EHL | HMM | KTY | DCT | RRL | RGL | NDI |
| EHK | HMF | KTV | DQE | RRK | RGK | NDK |
| EHM | HMP | KWW | DQL | RRM | RGM | NDP |
| EHS | HMS | KWY | DQK | RRF | RGF | NDS |
| EHT | HMT | KWV | DQF | RRP | RGP | NDY |
| EHY | HMW | KYY | DQP | RRS | RGS | NDV |
| NCK | | | | | | |

When any of variables described herein are null (absent) then the tripeptides or residues are connected to one another by a bond.

Accordingly, as described herein, the QMC can validate the amount of the molecule of interest as well as whether the reaction(s) are being performed efficiently and reproducibly.

Accordingly, in some embodiments, methods of validating a reaction of a test sample are provided. In some embodiments, the method comprises reacting a test sample comprising a pH-indicating agent, a molecule of interest and a QMC with a propionylating agent and a digesting agent and introducing the reacted sample into a mass spectrometer, wherein if one or more peaks produced in the mass spectrometer attributed to the QMC are above a selected threshold the reaction is validated for the reacting step. In some embodiments, the reaction does not comprise a pH-indicating agent.

Examples of molecules of interest include peptides, nucleic acid molecules, polymers, and the like. The molecule of interest can be a molecule that can be modified prior to digestion or disassociation prior to or during mass spectrometry analysis. In the non-limiting example provided herein, the molecule of interest is a molecule that can be propionylated and also subjected to digestion (e.g. proteolytic cleavage). In some embodiments, the molecule is a histone protein. Histone proteins are known to be enriched in lysine residues. The lysine residues can be propionylated unless the histone groups have been subject to other modifications, such as acetylation or methylation (e.g., trimethylation). The propionylation can protect the histones' lysines from proteolysis or other proteolytic cleavage.

The propionylating agent can be any agent that is capable of propionylating a molecule of interest and/or the standard. In some embodiments, the propionylating agent is propionic anhydride. The digesting agent can be any agent that can digest a protein into smaller fragments. Examples of digesting agents include, but are not limited to, proteases. A non-limiting example of a protease is trypsin.

In some embodiments, the method further comprises quantifying the molecule of interest. Quantifying the molecule of interest can be done, for example, by utilizing the QMC as a quantifying standard. This is done, for, example by comparing the peaks attributed to the molecule of interest to the peaks attributed to the QMC. The total peak intensity of the molecule of interest and the standard can be compared to one another to determine the quantity of the molecule of interest. The absolute amount of the molecule of interest can also be determined where the QMC has repeats of domains that are digested when the test sample is exposed to a digesting agent.

As such, in some embodiments, the methods disclosed herein can comprise analyzing the molecule of interest by mass spectrometry.

Mass spectrometry is referred to throughout the present disclosure. This includes, but is not limited to any method or machine that can be used for mass spectrometry. Examples include, but are not limited to, MALDI direct inject, ESI, LC-MS, FTICR, and the like.

The reaction can be validated if, for example, the total measurement of each possible fragment produced in the mass spectrometer attributed to the QMC are at least 80% of the expected area under the curve of intensity versus elution time or at least 80% of the expected peak intensity. In some embodiments, the threshold is at least 81, 82, 83, 84, 85, 86, 87, 88, 8, 9, 90, 91, 92, 93, 94, 95, 96, 97, 98, 99, or 100%. In some embodiments, the threshold is from 80% to 100%. As discussed herein, the QMC can be used to quantitate the amount of sample that is both digested and modified.

To determine yield by using the QMC, the total measurement (TM) of each possible fragment of the QMC is determined by mass spectrometry. TM can be determined by such ways as the area under the curve of intensity vs. elution time or total intensity. The sum of TM for the following categories unmodified/undigested (I_{u/u}), modified/undigested (I_{m/u}), unmodified/digested (I_{u/d}), and modified/digested (I_{m/d}), is determined (Equation 1).

Σ_{TM}=I_{u/u}+I_{u/d}+I_{m/u}+I_{m/d}.

Unmodified means, for example, that the standard is not propionylated, phosphorylated, or dephosphorylated, or otherwise subject to a post-translational modification. Modified means, for example, that the standard is propionylated, phosphorylated, or dephosphorylated, or otherwise subject to a post-translational modification. Digested or undigested means that the standard is digested through cleavage or not. For example, a cleavage after a arginine residue. The total measurement can be then be used to determine the yield modification. Yield modification is the ratio (or percent, Fₘ) of the sum of modified categories divided by the total sum of all categories (Equation 2).

$${F}_{m} = \frac{{I}_{m / u} + {I}_{m / d}}{{\sum }_{TM}} .$$

Fraction digested (F_{d}) is ratio of the sum of all of digested categories divided by the total sum of all categories (Equation 3).

$${F}_{d} = \frac{{I}_{u / d} + {I}_{m / d}}{{\sum }_{TM}} .$$

Accordingly, in some embodiments, the reaction is validated for modification if Fₘ is at least 0.7, 0.8, 0.9, or 0.95. In some embodiments, the reaction is validated for modification if Fₘ is from 0.7 to 1.0. In some embodiments, the reaction is validated for digestion of F_{d} is at least 0.7, 0.8, 0.9, or 0.95. In some embodiments, the reaction is validated for modification if F_{d} is from 0.7 to 1.0. In some embodiments, the reaction is validated for all conditions if Fₘ and F_{d} are both at least 0.7, 0.8, 0.9, or 0.95. In some embodiments, the reaction is validated if Fₘ is at least 0.7, 0.8, 0.9, or 0.95. In some embodiments, the reaction is validated if F_{d} is at least 0.7, 0.8, 0.9, or 0.95. Accordingly, the methods disclosed herein can be used to determine the validity of a chemical modification, which can also be referred to as derivatization. The methods can also be used to determine the validity of digestion without reference to the modifications and vice versa.

As discussed herein, if the standard has multiple domains the standard can be used to measure absolute concentration of the molecule of interest by providing a standard curve. The standard can be a polymer (e.g. peptide) with repeated domains with each domain having a different number of repeats. For example if the polymer has two domains, A and B, the number of repeats of A and B are different. Concentration of the molecule of interest can then be determined by, for example, Equation 4,

$${C}_{x} = {M}_{x} \times {C}_{i} \frac{{I}_{x}}{{\sum }_{TM}} ,$$

where Cₓ, is the concentration of domain of interest, Mₓ is the multiplication factor or the number of times the domain is repeated in the polymer, Cᵢ, is the molar concentration of the total peptide added to the sample, Iₓ is the total measurement of a domain of interest. The Cₓ for each domain is determined and plotted as a function of its measurement from the machine. This can then be used as a standard curve for determining the concentration of unknowns or the molecule of interest according to known methods. Thus, the present disclosure provides a molecule, the QMC, that can be used to quantify a molecule of interest as well as provide information as to whether the reaction is valid or not. The ability of a single molecule to perform each of these functions would not have been predictable.

As discussed herein, in some embodiments, the test sample can comprise a pH-indicating agent. The pH-indicating agent can be a visual indicator that tells the user that the reaction is taking place under the proper indications without actually measuring the pH with a pH meter. In some embodiments, the pH-indicating agent is a chromophore. Examples of pH-indicating agents include, but are not limited to, o-cresolphthalein or α-naphtholphthalein. In some embodiments, the pH-indicating agent indicates when a solution is at a pH of about 8. In some embodiments, the test sample does not comprise a pH-indicating agent.

In some embodiments, the test sample is reacted with the propionylating agent and digesting agent simultaneously. In some embodiments, the test sample is reacted with the propionylating agent prior to being reacted with the digesting agent. In some embodiments, the test sample is reacted with the propionylating agent after being reacted with the digesting agent. Any propionylating agent can be used including, but not limited to, propionic anhydride. Additionally, any digesting agent can be used. In some embodiments, the digesting agent is a protease. The protease can be, for example, a serine protease. In some embodiments, the digesting agent is trypsin.

As described herein, the QMC can be used to compare experimental samples across platforms, users, machinery, and experiments performed at different times because the QMC can be used as an internal standard based upon the Q-ratio described herein. Accordingly, in some embodiments methods are provided for cross-validating a plurality of reactions. In some embodiments, the method comprises performing a first reaction, the first reaction comprising reacting a first test sample comprising a pH-indicating agent, a molecule of interest and a quantitative multiplexed control (QMC) with a propionylating agent and/or a digesting agent; performing a second reaction, the second reaction comprising reacting a second test sample comprising a pH-indicating agent, a molecule of interest and a quantitative multiplexed control (QMC) with a propionylating agent and/or a digesting agent. In some embodiments, upon performing the first and second reaction, the reactions are run through a mass spectrometry. The reactions are performed separately so that the QMC can be quantified and the Q-ratio can be calculated for each reaction. Therefore, in some embodiments, the method comprises calculating a Q-ratio of the QMC of the first reaction and a Q-ratio of the QMC of the second reaction; wherein if the Q-ratio of the first reaction and the Q-ratio of the second reaction are substantially the same the first and second reactions are cross-validated; or wherein if the Q-ratio of the first reaction and the Q-ratio of the second reaction are not substantially the same the first and second reactions are not cross-validated. When the reactions are cross-validated with one another the data and results from the samples can be compared to one another with a high degree of confidence. In some embodiments, the molecule of interest of the first reaction and the molecule of interest of the second reaction are the same. They can also be different. In some embodiments, the method further comprises comparing the results of the first and second reaction by normalizing the results to the Q-ratio of the first and second reaction. If the Q-ratio are different then the differences between the two can be taken into account to normalize the results of the first and second reaction.

In some embodiments, the Q-ratio as described herein and throughout is the sum of the signals from all forms of the QMC. In some embodiments, all forms of the QMC are initial, modified only, digested only, and modified and digested. In some embodiments, the forms of the QMC are the initial and modified only. In some embodiments, all forms of the QMC are initial and digested only. In some embodiments, all forms of the QMC are the initial form and modified and digested form.

Q-ratio's are substantially the same when they are identical or within ± about 10, 9, 8, 7, 6, 5, 4, 3, 2, 1, or 0.5% of one another.

In some embodiments, kits are provided for performing a method described herein. In some embodiments, the kit includes instructions for performing the methods. In some embodiments, the kit includes a QMC, including but not limited to one or more of the QMC's described herein. In some embodiments, the kit includes a pH-indicating agent. In some embodiments, the kit includes a propionylating agent. In some embodiments, the kit includes a base. In some embodiments, the kit includes an extraction buffer. In some embodiments, the kit includes a quenching reagent. In some embodiments, the kit includes ammonium bicarbonate. The kit can comprise one or more or none of the elements recited herein. The pH-indicating agent can be a colorimetric agent that indicates a pH of about 8.0. In some embodiments, the pH-indicating agent is o-Cresolphthalein or α-Naphtholphthalein. The different components of the kit can be included in one or more containers. In some embodiments, the kit does not comprise a pH-indicating agent.

The extraction buffer can be a buffer for creating cell extracts to isolate the molecule of interest. Any suitable extraction buffer can be used. In some embodiments, the extraction buffer comprises a non-ionic detergent. In some embodiments, the detergent is 4-(1,1,3,3-Tetramethylbutyl)phenyl-polyethylene glycol (Triton™ X 100). In some embodiments, the extraction buffer comprises a protease inhibitor. In some embodiments, the protease inhibitor is a serine protease inhibitor. In some embodiments, the protease inhibitor is phenylmethylsulfonyl fluoride. In some embodiments, the extraction buffer comprises a preservative to prevent bacterial or other spoilage. In some embodiments, the preservative is sodium azide.

The base in the kit can be a base suitable for performing the reaction to the molecule of interest. The base will vary based upon application and can be modified to suit the user's application. In some embodiments, the base is ammonium hydroxide. In some embodiments, the kit includes propionic anhydride, which can act as the propionylating agent.

The kit can also include a quenching reagent, which can be used to stop the reaction of the test sample. A non-limiting example of a quenching reagent includes, but is not is limited to, formic acid.

In some embodiments, data is generated using a mass spectrometer. The data can be transmitted to a server (remote or local) and analyzed to generate results for the user. The generated results can determine the Fₘ, F_{d}, and/or Cₓ as well as the concentration of the molecule of interest based upon the generated data and results. The data can also be used to generate a report that tells the user that the reaction or experiment is valid. The server can interface with the user, for example, through the internet or run on a local workstation or computer.

### EXAMPLES

### Example 1

### Analysis of Histones in a Cell Test Sample

A 100 mm Cell Culture Dish with approximately 1×10⁷ cells is treated with an extraction buffer (PBS containing 0.5% Triton X 100 (v/v), 2 mM phenylmethylsulfonyl fluoride (PMSF), 0.02% (w/v) NaN3). 5 μg of total protein is mixed with a QMC (QLAATKAARAAKTAALQ, SEQ ID NO: 1) to form a test sample. The test sample is treated with 2 μL propionic anhydride and then immediately 6 μL ammonium hydroxide (NH₄OH) is added. The pH is adjusted with additional ammonium hydroxide, if necessary, to about 8, which is monitored with a pH-indicating agent (o-Cresolphthalein or α-Naphtholphthalein). After propionylation, trypsin is added to a final concentration of about 1:20 to 1:100 trypsin to total protein (e.g., 1 uL of 0.1 mg/mL) and 30 μL 50 mM NH₄HCO₃ (ammonium bicarbonate). The sample is vortexed. The pH is adjusted through the addition of NH₄OH to about 8. The sample is incubated at 37° C. overnight. 3.5 μL 10% FA (Formic acid) is added to the test sample solution and is mixed well. The solution is transferred to autosampler vials for LC-MS analysis. The undissolved proteins are left behind. Vials can be stored at 4° C. until ready to run. The sample is analyzed by mass spectrometry and validated by analyzing the peak intensity of the peaks attributed to the QMC.

### Example 2

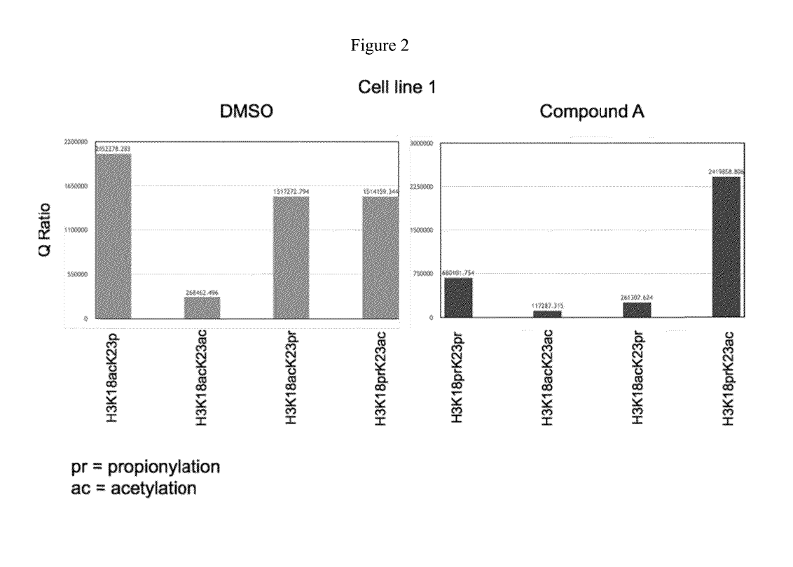

Ovarian cancer patient-derived cell lines that have been treated with DMSO (control) or 3 separate chemotherapeutic agents have been analyzed. After treatments, histones were extracted, chemically derivatized, and digested with trypsin protease. Following sample processing, quantitative QqQ and Orbitrap MS data was generated that elucidated histone lysine acetylation, methylation, or propionylation (unmodified) under different treatment conditions. The histones were analyzed according to Example 1. The QMC was analyzed in conjunction with histone samples, which allowed us to perform quantitative sample to sample as well as machine to machine comparisons. An example of the data generated is shown in FIG. 2.

### Example 3

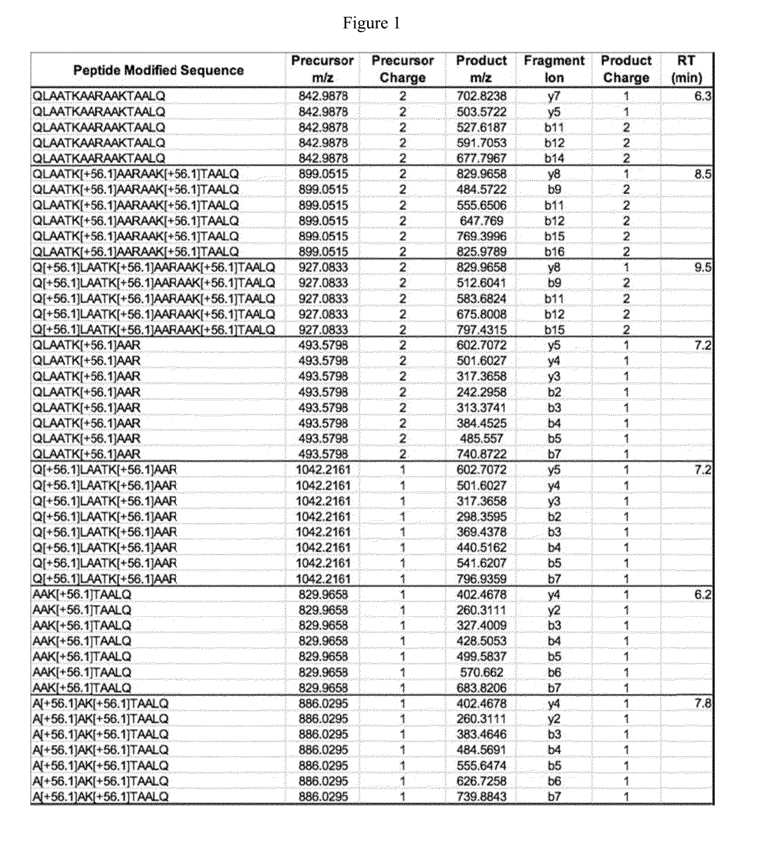

A QMC peptide with the sequence of QLAATKAARAAKTAALQ (SEQ ID NO: 1) was propionylated with propionic anhydride under conditions sufficient for propionylation (above pH 8). After the peptide was treated with propionic anhydride, the peptide was also digested with trypsin under conditions similar to those described in Example 1. The peptide and the reaction products were analyzed by mass spectrometry and column chromatography. The various fragments generated during the reaction are shown in FIG. 1. FIG. 1 demonstrates that a reaction can be monitored by mass spectrometry to determine the completeness of the digestion and the propionylation of the fragment. The peptide and its fragments were also quantified by column chromatography by eluting off of a C-18 column. The specific type of column is not critical and any suitable column could have been used (data not shown). Therefore, the ratio and amounts of the different fragments could be determined to validate the reaction. The fragments were detected using a nanoAcquity UPLC (Waters Corporation, Millford, Mass., USA) coupled with a Xevo TQ-S with ionKey Source. Two microliters of digested peptide sample (10 ng/mL) were injected and resolved using an iKey BEH C₁₈ 130, 1.7 im, 150 im×100 mm. Mobile phases were 0.1% Formic Acid in Water (A) and 0.1% Formic Acid in acetonitrile (B). Peptides were eluted over a 22 minute gradient of 5%-55% B at a flow rate of 3.10 iL/min. Total run time was 30 minutes. Data were acquired in positive ion mode at 3.5 kV with a source temperature of 120° C. MRM data was imported into Skyline v.2.5 (skyline.gs.washington.edu) for fragment ion annotation and peak area integration. Accordingly, the data demonstrated for the first time that a QMC peptide could be used to monitor the reaction and to validate analogous reactions.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

1. A method of validating a reaction of a test sample, the method comprising:
reacting a test sample comprising a pH-indicating agent, a molecule of interest and a quantitative multiplexed control (QMC) with a propionylating agent and/or a digesting agent to form a reacted sample; and
introducing the reacted sample into a mass spectrometer,
wherein if one or more peaks produced in the mass spectrometer attributed to the QMC are above a selected threshold the reaction is validated for the reacting step.

2. The method of claim 1, wherein the reacting step is a propionylation.

3. The method of claim 1, wherein the reacting step is a digestion.

4. The method of claim 1, the method further comprising quantifying the molecule of interest by comparing mass spectrometer peaks of the molecule of interest to the peaks of attributed to the QMC.

5. The method of claim 1, the method further comprising analyzing the molecule of interest by mass spectrometry.

6. The method of claim 1, wherein the reaction is validated if the total measurement of each possible fragment produced in the mass spectrometer attributed to the QMC is at least 80% of the expected area under the curve of intensity versus elution time or at least 80% of the expected peak intensity.

7. The method of claim 1, wherein the QMC is a peptide.

8. The method of claim 7, wherein the peptide is about 8 to about 20 residues.

9. The method of claim 1, wherein the QMC has a formula of R₁-X₁-R₂-X₂-R₃-X₃-R₄-X₄-R₅, wherein R₁, R₂, R₃, R₄, and R₅ are each independently a tripeptide or null provided that no more than two of R₁, R₂, R₃, R₄, and R₅ are null; and X₁, X₂, X₃, and X₄ are each independently null, lysine, arginine, or another residue that can be post-translationally modified, provided that at least one of X₁, X₂, X₃, and X₄ is lysine, arginine, or another residue that can be post-translationally modified.

10. The method of claim 1, wherein the QMC has a formula of: R₁-X₁-R₂-X₂-R₃-X₃-R₄, wherein R₁, R₂, R₃, and R₄, are each independently a tripeptide; and X₁, X₂, and X₃ are each independently null, lysine, arginine, or another residue that can be post-translationally modified, provided that at least one of X₁, X₂, and X₃, is lysine, arginine, or another residue that can be post-translationally modified.

11. The method of claim 1, wherein the QMC has a formula of: R₁-X₁-R₂-X₂-R₃, wherein R₁, R₂, and R₃, are each independently a tripeptide; and X₁ and X₂ are each independently null, lysine, arginine, or another residue that can be post-translationally modified, provided that at least one of X₁ and X₂ is lysine, arginine, or another residue that can be post-translationally modified.

12. The method of claim 6, wherein R₁, R₂, R₃, R₄, and R₅ are each independently selected from a tripeptide not found in nature.

13. The method of claim 9, wherein R₁, R₂, R₃, R₄, and R₅ are each independently selected from the group consisting of those listed in Table 1 and/or Table 2.

14. The method of claim 9, wherein R₁, R₂, R₃, R₄, and R₅ do not comprise a tripeptide selected from the group consisting of those listed in Table 3.

15. The method of claim 1, wherein the QMC comprises an amino acid sequence of QLAATKAARAAKTAALQ.

16. The method of claim 7, wherein the peptide comprises a plurality of domains, wherein the domains are separated by one or more digestion sites and/or by one or more post-translational modification sites.

17-18. (canceled)

19. The method of claim 16, wherein the peptide comprises a first domain, a second domain, and a third domain.

20. The method of claim 16, wherein the first domain, second domain, and third domain are independently selected from the group consisting of those listed in Table 1 and/or Table 2.

21-27. (canceled)

28. The method of claim 1, further comprising determining the concentration of the molecule of interest in the test sample by comparing the concentration of the peaks attributed to the QMC with the peak intensity attributed to the molecule of interest.

29. A method of cross-validating a plurality of reactions, the method comprising:
performing a first reaction, the first reaction comprising reacting a first test sample comprising a pH-indicating agent, a molecule of interest and a quantitative multiplexed control (QMC) with a propionylating agent and/or a digesting agent;
performing a second reaction, the second reaction comprising reacting a second test sample comprising a pH-indicating agent, a molecule of interest and a quantitative multiplexed control (QMC) with a propionylating agent and/or a digesting agent;
performing a first mass spectrometry run with the first reaction and a second mass spectrometry run with the second reaction;
calculating a Q-ratio of the QMC of the first reaction and a Q-ratio of the QMC of the second reaction;
wherein if the Q-ratio of the first reaction and the Q-ratio of the second reaction are substantially the same the first and second reactions are cross-validated; or
wherein if the Q-ratio of the first reaction and the Q-ratio of the second reaction are not substantially the same the first and second reactions are not cross-validated.

30-31. (canceled)

32. The method of claim 29, the method further comprising comparing the results of the first and second reaction by normalizing the results to the Q-ratio of the first and second reaction.

33-34. (canceled)

35. A kit comprising:
a QMC;
a pH-indicating agent;
a propionylating agent;
a base; and
optionally an extraction buffer, a quenching reagent, ammonium bicarbonate, or any combination thereof.

36-45. (canceled)

46. A quantitative multiplexed control (QMC), wherein the quantitative multiplexed control is a peptide.

47. (canceled)

48. The QMC of claim 46 having a formula of R₁-X₁-R₂-X₂-R₃-X₃-R₄-X₄-R₅, wherein R₁, R₂, R₃, R₄, and R₅ are each independently a tripeptide or null provided that no more than two of R₁, R₂, R₃, R₄, and R₅ are null; and X₁, X₂, X₃, and X₄ are each independently null, lysine, arginine, or another residue that can be post-translationally modified, provided that at least one of X₁, X₂, X₃, and X₄ are lysine, arginine, or another residue that can be post-translationally modified.

49-60. (canceled)

61. A composition comprising the QMC of claim 46.
